(19) United States
(12) Patent Application Publication
(10) Pub. No.: US 2023/0042455 A1
(43) Pub. Date: Feb. 9, 2023

(54) COMPOSITION FOR WEIGHT MANAGEMENT OF A SUBJECT

(71) Applicant: MRS-BIOTECH, Nîmes (FR)

(72) Inventor: Olivier MARÇAIS, Nîmes (FR)

(21) Appl. No.: 17/757,848

(22) PCT Filed: Dec. 28, 2020
(86) PCT No.: PCT/EP2020/087936

§ 371 Date: Jun. 22, 2022

(30) Foreign Application Priority Data
Dec. 31, 2019 (FR) 1915796

(51) Int. Cl. A61K 31/728; A61K 9/00; A61K 45/06

(52) U.S. Cl. CPC A61K 31/728; A61K 9/0019; A61K 45/06

(57) ABSTRACT

A composition for managing the weight of a subject, wherein the composition includes at least one high-viscosity, resorbable, biocompatible material, intended to be injected into the submucosal space of the digestive tract of a subject and thus creating a long-lasting raising of the corresponding mucosa is disclosed. Also disclosed is a kit including a composition according to the invention and structure for injecting the composition. Finally, the disclosure also relates to the use of a composition according to the invention for managing the weight of a subject.

### FIELD OF THE INVENTION

The subject of the present invention is a composition comprising a sterile solution of a resorbable biocompatible material for use in the weight management of a subject and for preventing or treating excess weight or obesity. The invention is therefore in the field of compositions for therapeutic use.

### STATE OF THE ART

Overweight, or excess weight, and obesity are defined by the World Health Organization as abnormal or excessive body fat accumulation. For an adult, overweight is when the body mass index (BMI) is greater than 25 kg/m², obesity is when the BMI is greater than 30 kg/m² and morbid obesity is when the BMI is greater than 40 kg/m². Obesity is a major global public health problem which is responsible for many morbid pathologies including in particular diabetes, hypertension, cardiopathies and osteoarticular deterioration. The incidence of many cancers is proportional to the BMI. Moreover, in the recent global pandemic due to the SARS COV2 virus (COVID 19), obesity emerged as one of the major independent risk factors for morbidity, for respiratory complications often requiring mechanical respiratory life support, and for death, together with diabetes and hypertension.

Various therapeutic approaches have been proposed in the last 30 years, which to date have had no noticeable effect: 99% of patients who follow a diet put weight back on over time.

Bariatric surgery has proved its worth in weight control and the reduction of associated co-morbidities, in particular diabetes, but at the expense of a significant morbidity ranging from 15 to 20% side effects and of a mortality of up to 1% in subjects operated on. The number of bariatric surgical operations is increasing exponentially every year, yet 99% of obese patients that are eligible for a bariatric operation do not have the operation for fear of the side effects of the surgery and the risk of putting weight back on over time.

Interventional and therapeutic gastrointestinal endoscopy is commonly carried out to access the digestive tract in order to treat lesions. Among the minimally invasive procedures to aid weight loss, placing an inflatable biocompatible device, or intragastric balloon, in the fundic cavity via endoscopy has been proposed in the last 30 years. Even so, these procedures do not meet with the support of the public, who fear their side effects.

Minimally invasive procedures to aid the weight management of a subject, which are considered to be low-risk, simple and quick to carry out, easy to learn and of a reasonable cost, are therefore highly desirable and very long-awaited. Compositions intended for the implementation of said procedures are therefore also long-awaited.

### STATE OF THE ART

The present invention meets these objectives. The inventor has in fact developed injectable compositions comprising a solution of at least one bioresorbable biocompatible biomaterial, which are intended to be used in a process comprising the administration of an effective quantity of said injectable composition into the submucosa of the gastrointestinal tract, via an endoscopic procedure. Surprisingly, compositions according to the invention make it possible to create cushions generating a long-lasting elevation of the mucosa, for at least 3 h, preferably for at least 14 hours. Generating this elevation causes a reduction in the digestive lumen of from approximately 10% to 30%, or even more.

A composition according to the invention has the advantage of having a viscosity that is sufficient to make it possible to easily raise the portion of the digestive mucosa situated at the level of the submucosal injection, while being able to be manipulated during an endoscopic procedure. In particular, a composition according to the invention can be administered by means of a catheter with a length of from 100 to 200 cm and with a diameter of from 1.5 to 5 mm. Moreover, a composition according to the invention, due to its nature and its viscosity and, where appropriate, the crosslinking of the material that it contains, has a sufficient strength which enables it to raise the digestive mucosa concerned and to stay at the level of the injection site for a sufficient time when it is situated in the submucosal space of a portion of the digestive tract. Without being bound by a particular theory, this sufficient strength is constituted in particular by a mechanical strength in contact with the fibres of the submucosal space, and by a resistance to local enzymatic degradation.

The publication "Multicenter randomized study of obesity treatment with minimally invasive injection of hyaluronic acid versus and combined with intragastric balloon" by J. Dargent et al. (Obesity Surgery 2015, 25: 1842-7) describes a process, combined with placing a gastric balloon, comprising the administration via endoscopy at the gastro oesophageal junction to obese patients of a solution of low-viscosity linear hyaluronic acid that is largely used in orthopaedic surgery: sodium hyaluronate (sodium salt of hyaluronic acid) with a molecular weight of 800 kDa at a concentration of 0.8% or 8 mg hyaluronic acid per 1 ml of buffered physiological sodium chloride solution (Sinovial™, Genevrier, France). The authors observe that "the injection of hyaluronic acid is ineffective compared with the placing of an intragastric balloon for inducing weight loss" (p. 1846, col. G) and conclude that the efficacy of injections of hyaluronic acid in the treatment of obesity is not demonstrated. In their conclusion, the authors discourage the implementation of such an approach unless it proves more effective than the existing approaches, which is not the case in this publication.

EP 1 352 661 describes the use, for the endoscopic resection of a portion of mucosal tissue, of saline solutions comprising hyaluronic acid with a molecular weight of from 1,500 to 3,000 kDa at a concentration comprised between 0.1% and 0.7%, in a 0.8 to 1% saline solution. The submucosal injection of these solutions leads to an elevation of the mucosa for a duration of 90 minutes.

WO 2015/019 304 describes the use of a composition comprising a polysaccharide and hyaluronic acid for its use as a medicament for the treatment of articular pathologies in particular, the injection of which is carried out using a syringe. The state of the art does not describe compositions according to the invention, which are used to generate a long-lasting elevation of the submucosa of the gastrointestinal tract for at least 3 hours, preferably for at least 14 hours, in order to induce a weight loss or to modify food intake.

### DISCLOSURE OF THE INVENTION

With respect to the compositions of the state of the art, a composition according to the invention makes it possible for a treated subject to observe, after a simple endoscopic procedure, a significant and long-lasting weight loss. This weight loss, of from 5 to 10% of the total body mass can in general reach 20% or more of the total body mass, or a minimum of 30 to 50% of the excess body weight. Surprisingly, a composition according to the invention, administered to a subject, causes a decreased appetite, a lowering of the food intake and/or volume, earlier satiety, and/or a reduction in thoughts about food.

A composition according to the invention therefore presents a safe method, devoid of side effects, which is simple and quick to carry out, easy to learn, reversible and of a reasonable cost and which makes it possible to treat excess weight and to prevent or treat obesity and morbid obesity.

### DESCRIPTION OF THE FIGURE AND THE EMBODIMENTS

Other advantages and characteristics will become apparent on examination of the detailed description of an embodiment which is in no way limitative, and from the attached drawings, in which:

FIG. 1 is a diagrammatic representation of the progression of the elevation of a mucosa in a pork belly model, as a function of time after the injection of compositions according to the invention.

Of course, the embodiments that will be described hereinafter are in no way limitative. Variants of the invention can in particular be imagined comprising only a selection of the characteristics described hereinafter, in isolation from the other characteristics described, if this selection of characteristics is sufficient to confer a technical advantage or to differentiate the invention with respect to the state of the prior art.

### DETAILED DESCRIPTION OF THE INVENTION

According to a first subject, the present invention relates to an injectable composition for use thereof in the weight management of a subject, comprising a high-viscosity isotonic saline solution of at least one biocompatible and resorbable material, said composition being intended to be used in a process which comprises the administration of an effective quantity of said injectable composition under the mucosa of the gastrointestinal tract, during an endoscopic procedure, in order to obtain a long-lasting protrusion of said mucosa for at least 3 hours, preferably for at least 14 hours.

By "weight management" is meant aiding weight loss in the therapeutic treatment of excess weight, in the prevention and/or treatment of obesity and morbid obesity. The treated subject is preferably a mammal, preferably a human.

By "viscosity" is meant all of the flow resistance phenomena that occur in the mass of a material, for a flow that is uniform and without turbulence. For a composition according to the invention, the suitable viscosity must be sufficiently high to make it possible to generate a long-lasting elevation of the mucosa at the injection site(s) and compatible with the administration of said composition during an endoscopic procedure, by means of a catheter-type device, preferably a catheter. The viscosity is in particular proportional to the molecular weight, expressed in daltons (Da) or kilodaltons (kDa), of the polymer and to its concentration in the composition. By "high-viscosity" is meant a composition according to the invention which has a viscosity comprised between 0.01 Pa.s (pascal second) and 40 Pa.s, preferably between 0.02 and 10 Pa.s while being compatible with a flow rate of from 0.1 to 0.8 ml/second through a catheter for endoscopic injection of 160 cm and a 19 G needle (Life Partner Europe) using a 3 ml syringe (Shangai Kindly Medical Instrument, ZSQ3B model). A person skilled in the art will easily choose a composition according to the invention the viscosity of which is suitable for injection by means of a catheter.

A composition according to the invention is characterized by: i) an elastic modulus G', G' being the shear storage modulus, in torsion, measured at 25° C. and at 1 Hz, comprised between 0.1 and 10 Pa, preferably between 0.2 and 8 Pa, ii) a viscous modulus G", G" being the shear loss modulus, in torsion, measured at 25° C. and at 1 Hz, comprised between 0.1 and 10 Pa, preferably 0.2 and 5 Pa and iii) a tan D to the plateau, tan D being the tangent of the phase angle, i.e. G"/G', of from 0.1 to 10, preferably 0.4 to 8.

According to a particular embodiment, the subject of the invention is therefore a sterile injectable composition for use thereof in the weight management of a subject, comprising an isotonic saline solution with a viscosity comprised between 0.01 Pa.s and 40 Pa.s, preferably comprised between 0.02 and 10 Pa.s, of at least one biocompatible and resorbable material.

By "biocompatible material" is meant a material that is capable of being accepted by a living being, without any adverse effect on the environment in which it is implanted.

With respect to a non-resorbable material, the resorbable characteristic of a material of a composition according to the invention makes it possible to avoid the undesired migration of the product and the formation of a granuloma in response to a foreign body. The products of a composition according to the invention are naturally and spontaneously resorbed in a defined time, without leaving a residual trace. In other words, by "resorbable material", or "bioresorbable material", is meant a material that is naturally and spontaneously resorbed without leaving a residual trace, after a period of from approximately 1 to 24 months, preferably of from 3 to 18 months.

By "submucosa of the gastrointestinal tract" is meant the space situated beneath the digestive mucosa, between the mucosa and the digestive muscularis, in the space called submucosal, for example of the oesophagogastric or antropyloric or oesophageal-cardiac junction, or of a digestive anastomosis. This space is constituted by a framework of connective tissue with elastic fibres, collagen, blood vessels, sympathetic nerves comprising Meissner's plexus and sometimes secretory glands. A composition according to the invention can be injected at one or more sites of the digestive submucosa, for example of the oesophagogastric junction. An endoscopic procedure for administering a composition according to the invention is a conventional endoscopic procedure that is well known to a person skilled in the art. A volume of from 0.5 to 5 ml, preferably of from 0.5 to 2 ml, preferably 1 ml, of a composition according to the invention can be administered per puncture or injection site. An elevation of from 0.1 to 5 cm, preferably of from 0.1 to 2 cm, preferably of from 0.4 to 1 cm is obtained for each injection site. A multiple puncture technique makes it possible to produce a ridge, which can be circumferential, on one or more levels.

According to a particular embodiment of a composition according to the invention, in an ex vivo model of a portion of pork belly, after injection of a composition according to the invention into the submucosa of the gastrointestinal tract at least 50% of the height of the initial elevation of the mucosa is still present 14 hours after said injection, and preferably at least 50% of the height of the initial elevation of the mucosa is still present 98 hours after said injection. The subject of the invention is therefore an injectable composition for use thereof in the weight management of a subject, comprising a high-viscosity isotonic saline solution of at least one biocompatible and resorbable material, said composition being intended to be used in a process which comprises the administration of an effective quantity of said injectable composition under the mucosa of the gastrointestinal tract, during an endoscopic procedure.

According to a more particular embodiment of a composition according to the invention, said at least one biocompatible material is a polymer selected from: i) polysaccharides and polysaccharide derivatives, in particular hyaluronic acid, cellulose and its derivatives, starch, alginic acid or one of its salts, chondroitin and chitosan; ii) polycaprolactone; iii) polyglycolic acid, polylactic acid, homopolymers of lactic acid, iv) purified inverse thermosensitive polymers and v) a mixture thereof. In a composition according to the invention, said polymers are present in the form of an isotonic saline solution suitable for the use of the composition. Said isotonic saline solution, or said isotonic saline solutions, is/are prepared by any means known to a person skilled in the art.

According to a particular embodiment, the subject of the invention is an injectable composition for use thereof in the weight management of a subject, comprising an isotonic saline solution with a viscosity comprised between 0.01 Pa.s and 40 Pa.s, preferably comprised between 0.02 and 10 Pa.s, of at least one polymer selected from polysaccharides and polysaccharide derivatives.

According to an even more particular embodiment, the subject of the invention is an injectable composition for use thereof in the weight management of a subject, comprising an isotonic saline solution with a viscosity comprised between 0.01 Pa.s and 40 Pa.s, preferably comprised between 0.02 and 10 Pa.s, of at least one polysaccharide, or polysaccharide derivative, said polysaccharide or polysaccharide derivative being crosslinked.

By "solution of at least one crosslinked polysaccharide or polysaccharide derivative" is meant a solution in which the molecules of said at least one polysaccharide, or polysaccharide derivative, are bridged by intramolecular bonds. The degree of crosslinking of a solution, expressed as a percentage, is the ratio between the number of moles of molecules of said at least one polysaccharide and the number of moles of molecules of the crosslinking agent, multiplied by 100. In a solution according to the invention, a suitable biocompatible crosslinking agent can easily be selected by a person skilled in the art from the various known crosslinking agents.

According to an even more particular embodiment, the subject of the invention is therefore an injectable composition for use thereof in the weight management of a subject, comprising an isotonic saline solution with a viscosity comprised between 0.01 Pa.s and 40 Pa.s of at least one polysaccharide, or polysaccharide derivative, said polysaccharide or polysaccharide derivative being crosslinked, with a degree of crosslinking comprised between 1 and 40%.

Among the polysaccharides, the cellulose derivatives that may be mentioned are: methyl cellulose, carboxymethyl cellulose and hydroxypropyl cellulose at a concentration of from 0.2 to 1% (mass/volume).

Alginic acid, or one of its salts such as sodium alginate, can be present at a concentration of from 0.3 to 3% with respect to the volume of the composition according to the invention.

Polycaprolactone (PCL) is constituted by polymerized hydroxycaproic acid, its molecular weight is comprised between 100 and 1,500 ,000 g/ml, it is present at a concentration of from 1 to 2.5%, the particle diameter of PCL is in particular around 25 to 50 µm.

Among the homopolymers of lactic acid, there may be mentioned for example poly-L-lactic acid, poly-D-lactic acid, its copolymers and compositions thereof. Inverse thermosensitive polymers are sequenced copolymers of polyoxyalkylene which are present at a concentration of from 5 to 30% (mass/volume). Poloxamer P407 and poloxamer P188 may be mentioned in particular.

In a composition according to the invention comprising chitosan, the latter is present at a concentration of from 0.2 to 1% (mass/volume).

According to a more particular embodiment of a composition according to the invention, said at least one polymer is hyaluronic acid or a salt thereof, with a molecular weight of greater than 800 kDa, preferably comprised between 900 and 4,000 kDa, preferably comprised between 900 and 3,500 kDa, preferably between 1,200 and 2,400 kDa, preferably between 1,200 and 1,600 kDa, preferably between 1,250 and 1,600 kDa. In the present description, the limits of the value ranges are included in said value range.

According to an even more particular embodiment, said at least one polymer is hyaluronic acid or a salt thereof, with a molecular weight of greater than 800 kDa and less than 1,000 kDa, and with a hyaluronic acid concentration comprised between 0.2 and 1%.

According to another even more particular embodiment, said at least one polymer is hyaluronic acid or a salt thereof, with a molecular weight comprised between 900 and 4,000 kDa, preferably comprised between 900 and 3,500 kDa, preferably between 1,000 and 2,400 kDa, preferably between 1,200 and 2,400 kDa, preferably between 1,200 and 1,600 kDa, preferably between 1,250 and 1,600 kDa, and with a hyaluronic acid concentration comprised between 0.1 and 0.8%. By "hyaluronic acid" is meant a polysaccharide that is formed by repeating a disaccharide unit constituted by D-glucuronic acid and D-N-acetylglucosamine.

According to an even more particular embodiment of a composition according to the invention, said at least one polymer is linear hyaluronic acid, crosslinked hyaluronic acid, modified hyaluronic acid or a combination thereof.

According to an even more particular embodiment of a composition according to the invention, said at least one polymer is linear hyaluronic acid, or a salt thereof, with a molecular weight of greater than 800 kDa, preferably comprised between 900 and 4,000 kDa, preferably between 900 and 3,500 kDa, preferably between 1,200 and 2,400 kDa, preferably between 1,200 and 1,600 kDa. When the forms of hyaluronic acid are in the form of salts, these are preferably sodium salts.

According to an even more particular embodiment of a composition according to the invention, said at least one polymer is crosslinked hyaluronic acid, or a salt thereof, with a molecular weight of greater than 800 kDa, preferably comprised between 900 and 4,000 kDa, preferably between 900 and 3,500 kDa, preferably between 1,200 and 2,400 kDa, preferably between 1,200 and 1,600 kDa, preferably between 1,250 and 1,600 kDa. Crosslinked hyaluronic acid is obtained by bringing hyaluronic acid and a crosslinking agent together, leading to the formation of a covalent bond between two or more hyaluronic acid polymer chains. In a composition according to the invention, hyaluronic acid is crosslinked with a degree of crosslinking of from 0.0001 to 0.5, preferably 0.01 to 0.4, preferably 0.02 to 0.2. In other words, the degree of crosslinking of a composition according to the invention is therefore from 0.01 to 50%. More particularly, the degree of crosslinking of a composition according to the invention is preferably 1 to 40%, preferably 1.5 to 40%, preferably 1.5 to 30%, preferably 2 to 20%.

Modified hyaluronic acid is obtained by covalent substitution of hyaluronic acid, for example grafting an amino acid onto the -COOH group of hyaluronic acid, which can make it possible to increase its lifespan in the tissues. A composition according to the invention has a lifespan in the tissues of between 3 and 24 months, more particularly between 6 and 18 months.

According to an even more particular embodiment of a composition according to the invention, said at least one polymer is linear hyaluronic acid with a molecular weight of greater than 800 kDa, preferably comprised between 900 and 4,000 kDa, preferably comprised between 900 and 3,500 kDa, preferably between 1,200 and 2,400 kDa, preferably between 1,200 and 1,600 kDa, preferably between 1,250 and 1,600 kDa.

According to an even more particular aspect of a composition according to the invention, said composition comprises at least a first solution of a first polymer, preferably linear hyaluronic acid with a molecular weight of greater than 800 kDa, and a second solution of a second polymer which is different from linear hyaluronic acid. A preferred composition according to the invention comprises at least: i) a solution of linear hyaluronic acid with a molecular weight of greater than 800 kDa, and ii) a solution of crosslinked hyaluronic acid with a molecular weight of greater than 800 kDa, or a solution of polysaccharide or a solution of a polysaccharide derivative.

According to an even more particular aspect of a composition according to the invention, said composition comprises at least a first solution of a first polymer, preferably linear or crosslinked hyaluronic acid, with a molecular weight of greater than 800 kDa, preferably comprised between 900 and 4,000 kDa, preferably comprised between 900 and 3,500 kDa, preferably between 1,200 and 2,400 kDa, preferably between 1,200 and 1,600 kDa, preferably between 1,250 and 1,600 kDa, and a second solution of a second polymer, preferably linear or crosslinked hyaluronic acid, with a molecular weight of greater than 800 kDa, preferably comprised between 900 and 4,000 kDa, preferably comprised between 900 and 3,500 kDa, preferably between 1,200 and 2,400 kDa, preferably between 1,200 and 1,600 kDa, preferably between 1,250 and 1,600 kDa, said first and second solutions themselves potentially being combined by means of an additional level of crosslinking (double crosslinking).

According to another particular embodiment of a composition according to the invention, said solution comprises between 0.05% and 1.5% hyaluronic acid, preferably between 0.1% and 1.2% hyaluronic acid, preferably between 0.2% and 1% hyaluronic acid, preferably between 0.4% and 1%, preferably between 0.4% and 0.8%, the percentage being expressed as a mass by volume of composition. A concentration of 1% hyaluronic acid is equivalent to 10 mg hyaluronic acid in 1 ml of composition.

According to an even more particular embodiment, said at least one polymer is hyaluronic acid or a salt thereof, with a molecular weight of greater than 800 kDa and less than 1,000 kDa, and with a hyaluronic acid concentration comprised between 0.2% and 1%, preferably between 0.4% and 1%.

According to another even more particular embodiment, said at least one polymer is hyaluronic acid or a salt thereof, with a molecular weight comprised between 1,000 and 2,400 kDa, preferably between 1,200 and 2,400 kDa, preferably between 1,200 and 1,600 kDa, preferably between 1,250 and 1,600 kDa, and with a hyaluronic acid concentration comprised between 0.1% and 1.2% hyaluronic acid, preferably between 0.2% and 1%, preferably between 0.4 and 0.8%.

Even more particularly, a composition according to the invention comprises hyaluronic acid or a salt thereof, and a polymer selected from: i) polysaccharides and polysaccharide derivatives, in particular hyaluronic acid, cellulose and its derivatives, starch, alginic acid or one of its salts, chondroitin and chitosan; ii) polycaprolactone; iii) polyglycolic acid, polylactic acid, homopolymers of lactic acid, iv) purified inverse thermosensitive polymers and v) a mixture thereof. An even more particular composition according to the invention comprises linear hyaluronic acid or a salt thereof, and crosslinked hyaluronic acid or a salt thereof.

According to another particular embodiment of a composition according to the invention, said composition comprises between 1 and 50 mg/ml hyaluronic acid, preferably between 2 and 40 mg/ml, between 3 and 20 mg/ml or between 3 and 10 mg/ml.

According to another particular embodiment, a composition according to the invention also contains an inert mineral substance, such as hydroxyapatite or calcium triphosphate, in a suitable quantity. According to a more particular aspect, a composition according to the invention comprises hydroxyapatite particles with a suitable diameter and in a suitable concentration, in combination with a polymer. According to another particular aspect of a composition according to the invention, said at least one resorbable biocompatible material is dissolved in an aqueous saline solution containing from 0.8% to 1% sodium chloride, expressed as a weight with respect to the volume of the solution.

According to another particular aspect of a composition according to the invention, said composition also comprises at least one agent selected from: an anaesthetic agent, a vasoconstrictor agent, a colouring agent and an antioxidant.

In a composition according to the invention, an anaesthetic agent is selected from the anaesthetic agents known to a person skilled in the art, and in particular lidocaine, marcaine, mepivacaine and bupivacaine.

In a composition according to the invention, a vasoconstrictor agent is selected from the vasoconstrictor agents known to a person skilled in the art, and in particular epinephrine and noradrenaline.

In a composition according to the invention, a colouring agent is selected from the colouring agents known to a person skilled in the art, for example indigo carmine and methylene blue. The presence of a colouring agent makes it possible to observe the elevation and to ensure good diffusion of the product into the digestive submucosal space concerned.

In a composition according to the invention, an antioxidant agent is selected from the antioxidant agents known to a person skilled in the art, and in particular polyols, vitamin C, magnesium and vitamin E. The presence of an antioxidant agent has the effect of protecting the material from attack by free radicals, thus limiting its degradation and extending its lifespan in the tissues.

According to a second subject, the present invention relates to a kit comprising at least one injectable composition according to the invention, preferably contained in at least one syringe, at least one ampoule or at least one sterile bag, a sterile single-use catheter-type device can be combined in the same kit or in a separate kit. The syringe(s), ampoule(s) and/or sterile bag(s) contains from 1 to 20 ml of composition according to the invention. The catheter intended to produce the elevations of the mucosa has a length and a diameter that are adapted to the endoscopic apparatus used to carry out the administration of said composition, its length being comprised between 1,000 and 2,500 mm, between 1,200 and 1,800 mm, and its diameter between 1.5 and 5 mm, preferably between 1.8 and 4 mm, preferably between 2 and 3.8 mm, with a needle with a length of from 2 to 10 millimetres, preferably of from 4 to 8 mm, preferably of from 4 to 6 mm, with a 25 to 19 gauge diameter (0.5 to 1.2 mm). Said catheter-type device can be an endoscopic sclerotherapy needle or an endoscopic submucosal injection needle which is adapted to the size of the endoscope used. The catheter can in particular be positioned using an endoscope.

According to a particular aspect, a kit according to the invention also comprises a traceability device, for example detachable labels, and/or instructions for use in at least one language. A device for aiding positioning of the product, such as for example a high-pressure device may be available in the same kit or in a separate kit.

According to a third subject, the present invention relates to the use of an injectable composition according to the invention for increasing satiety, for limiting the feeling of hunger, for treating excess weight, for preventing or treating obesity, for managing excess fat or for managing the weight of a subject.

According to a particular aspect, a use according to the invention is intended to treat a subject suffering from excess weight, obesity or morbid obesity. More particularly, a use according to the invention is intended to prevent and/or treat the morbid complications of obesity, such as in particular diabetes, hypertension, cardiopathies, dyslipidemia, hepatic metabolic syndrome (steatohepatitis) and gastroesophageal reflux.

According to a fourth subject, the present invention relates to a method for preventing or treating a subject suffering from excess weight, obesity or morbid obesity, said method comprising the administration of an effective quantity of a composition according to the invention into the submucosa of the gastrointestinal tract of the subject, during an endoscopic procedure.

More particularly, a treatment method according to the invention is intended to prevent and/or treat the morbid complications of obesity, such as in particular diabetes, hypertension, cardiopathies, dyslipidemia, hepatic metabolic syndrome and gastroesophageal reflux.

According to a particular aspect, the subject of the invention is a method for treating a subject suffering from excess weight or whose body mass index (BMI) is greater than 25 kg/m². According to another particular aspect, the subject of the invention is a method for treating a subject suffering from obesity or whose BMI is greater than 30 kg/m². According to another particular aspect, the subject of the invention is a method for treating a subject suffering from morbid obesity or whose BMI is greater than 40 kg/m².

Examples of compositions according to the invention are compiled in Table 1 below.

Table 1

| Compound | Molecular weight (kDa) | Quantity of hyaluronate (mg) | Concentration (%) | Volume of solution (ml) |
|---|---|---|---|---|
| Linear sodium hyaluronate (NaHA) | 1,200 | 100 | 1 | 10 |
| | 800 and 1,200 ( ⅓-⅔) | 64 | 0.8 | 8 |
| | 1,600 | 100 | 1 | 10 |
| | 1,200 | 64 | 0.8 | 8 |
| | 2,400 | 100 | 1 | 10 |
| | 3,500 | 60 | 0.6 | 10 |

Other advantages and characteristics of the invention will become apparent on examination of the detailed description of an embodiment which is in no way limitative, and from the attached figure.

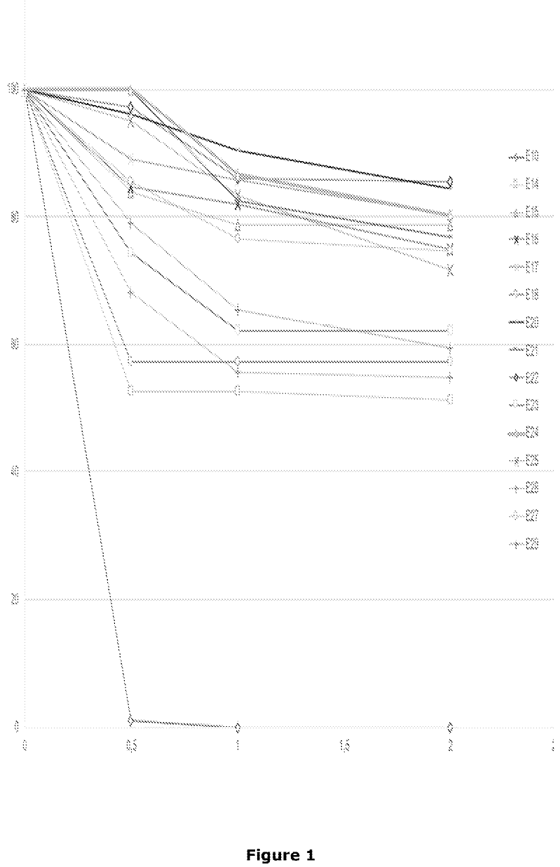

FIG. 1 represents the progression of the elevation of a mucosa in a pork belly model, in which the x axis represents the time (in days) and the y axis represents the height of the mucosa measured at 0.5, 1 or 2 days, expressed as a percentage with respect to the height of the initial elevation (height % = 100 x(height of the mucosa elevation in mm at T0 - height of the mucosa elevation at Tx)/height at T0.

The present invention will be better understood on reading the following examples, which are given in order to illustrate the invention and not to limit the scope thereof.

### EXAMPLES

### Example 1: Determination of the Elevation of a Mucosa After Injection of Compositions According to the Invention

It is well known that measurement of the progression of the submucosal injections carried out ex vivo constitutes a good reflection of what is happening in vivo and is therefore a reliable analysis tool. The objective of the present example is to measure, at ambient temperature, in a mucosa or portion of pork belly model, the elevation of said mucosa following the submucosal injection of a composition of the prior art and of compositions according to the invention. In a first series of experiments, the elevations were measured over 60 minutes and then, in a second series of experiments, the elevations were measured over 14 hours.

Compositions E10 to E29 are defined in Table 2 below.

Table 2

| | |
|---|---|
| Reference | Composition |
| E10 | Sodium hyaluronate (NaHA) solution 800 kDa at 0.8% (Sinovial™, laboratoire Genevrier) |
| E14 | Chitosan solution (0.99 g 0.5% chitosan (Kitozyme) 1200 kDa), 5% crosslinked (viscosity 5 to 35 Pa.s) |
| E15 | Homogeneous mixture of 70 parts NaHA solution 300 kDa 7.5 mg/ml 6% crosslinked and 30 parts NaHA solution 3,000 kDa 5 mg/ml 6% crosslinked Mannitol 3% Lidocaine 0.3% |
| E16 | Homogeneous mixture of 33 parts NaHA solution 1,500 kDa 5 mg/ml 12% crosslinked and 67 parts NaHA solution 1,500 kDa 5 mg/ml uncrosslinked Sorbitol 3% Lidocaine 0.3% |
| E17 | Homogeneous mixture of 25 parts NaHA solution 1,500 kDa 5 mg/ml 9% crosslinked and 75 parts NaHA solution 300 kDa 3.3 mg/ml 27% crosslinked Sorbitol 3% Lidocaine 0.3% |

| | |
|---|---|
| E18 | Homogeneous mixture of 60 parts NaHA solution 1,500 kDa 4 mg/ml 12% crosslinked and 40 parts NaHA solution 3,000 kDa 3 mg/ml 8% crosslinked Mannitol 3% Lidocaine 0.3% |
| E20 | Homogeneous mixture of 70 parts 1% Carboxy Methyl Cellulose (CMC) solution 8% crosslinked with hydroxyapatite and 30 parts NaHA solution 3,000 kDa 3 mg/ml 8% crosslinked |
| E21 | Homogeneous mixture of 50 parts 0.6% NaHA solution 2,700 kDa, 35 parts 1% CMC 6% crosslinked and 15 parts hydroxyapatite |
| E22 | Homogeneous mixture of 90 parts NaHA solution 2,600 kDa 5 mg/ml and 10 parts NaHA solution 300 kDa 5 mg/ml 19% crosslinked |
| E23 | Homogeneous mixture of an NaHA solution 2,600 kDa 4 mg/ml 19% crosslinked |
| E24 | Homogeneous mixture of 4 parts NaHA solution 300 kDa 5 mg/ml 19% crosslinked and 96 parts NaHA solution 1,200 kDa 5 mg/ml |
| E25 | Homogeneous mixture of 32 parts NaHA 1,200 kDa free 4 mg/ml, 8 parts NaHA 1,200 kDa 2.5 mg/ml, 26% crosslinked and 60 parts NaHA 300 kDa 4 mg/ml |
| E27 | Homogeneous mixture of 50 parts NaHA 1,500 kDa 4 mg/ml 12% crosslinked and 50 parts NaHA 1,500 kDa 4 mg/ml 6% crosslinked, said mixture is then 3% crosslinked in the presence of BDDE Sorbitol 3% Lidocaine 0.3% |
| E28 | Homogeneous mixture of 50 parts NaHA 1,500 kDa 3.3 mg/ml 13% crosslinked and 50 parts NaHA 1,500 kDa 3.3 mg/ml 5% crosslinked, said mixture then being 1% crosslinked in the presence of BDDE Mannitol 3% Lidocaine 0.3% |
| E29 | Homogeneous mixture of 50 parts NaHA 1,500 kDa 2.5 mg/ml 13% crosslinked and 50 parts NaHA 1,500 kDa 2.5 mg/ml uncrosslinked Sorbitol 3% Lidocaine 0.3% |

The compositions are prepared according to protocols that are well known to a person skilled in the art. Fibres or a sodium hyaluronate (NaHA) powder are hydrated in an alkaline NaOH solution and mixed with a spatula at a temperature of from 12 to 25° C. until a homogeneous alkaline sodium hyaluronate solution is obtained.

Composition E27, double-crosslinked, is prepared as follows: a cohesive homogeneous single-phase mixture of 0.4% NaHA 1,500 kDa, double-crosslinked, is produced by mixing: gel A comprising 0.9 g NaHA of injectable quality 1,500 kDa in 6.5 ml 1% NaOH, mix with a spatula for 1 h at 25° C., add 60 mg BDDE as a 20% dilution in NaOH in order to obtain a crosslinking characterized by R=12%; and gel B comprising 0.9 g injectable NaHA 1,500 kDa in 6.5 ml 1% NaOH, mix with a spatula for 1 h at 25° C., add 30 mg BDDE as a 20% dilution in NaOH in order to obtain a crosslinking characterized by R=6%. Then, the product is prepared by mixing the two hydrogels A and B with a spatula for 15 min, adding 30 mg BDDE (butanediol diglycidyl ester) as a 20% dilution in 0.25 N NaOH, mixing with a spatula at ambient temperature, placing over a bain marie at 50° C. for 1 hour, adding HCl dropwise to pH 7.

The ex vivo analysis technique is as described by H. Al-Taie et al. in 2012 ("Efficacy of submucosal injection of different solutions inclusive blood components on mucosa elevation for endoscopic resection", Clinical and Experimental Gastroenterology, 2012, vol. 5, pp. 43-48) with regard to the efficacy of different components in creating a submucosal cushion up to 60 minutes after they have been injected in order to optimize the endoscopic resection of digestive mucosal walls. Square 50 x 50 mm portions of pork belly are cut out and fixed at their four corners with needles on 50 x 50 mm cork sheets, at a temperature of 25° C. A specific mounting was created in order to obtain a measuring distance and a constant viewing angle of the samples with respect to the camera. The camera is mounted on a tripod and fixed to a flat support. The support for the cork sheets is fixed on a work surface at a suitable height. A sheet of squared paper is fixed behind each sample, making it possible to calibrate the measurement when using analytical software. 1 ml of each of the compositions is injected tangentially into the submucosal space of the pork belly samples fixed beforehand, using a 21 G needle. Photographs are taken of each of the pork belly portions immediately after injection and then every 5 minutes for 1 hour. Each measurement is repeated 3 times independently and blind. The images are analyzed using Adobe Illustrator (v C33, Adobe System Inc., San Jose, CA) software, which measures the mucosa elevation with an accuracy of ⅟100 mm before and after injection. A control is effected by means of the injection of physiological serum.

A clear and rapid collapse of the elevation of the mucosae is observed after injection of the physiological serum. A rapid collapse of the elevation of the mucosae is also noted after injection of sample 10 (E10) over the course of 60 minutes after injection: one hour after injection, less than 72% of the initial elevation is observed. For compositions 14 to 29, the elevation remains stable and unchanged: a 100 % maintenance of the elevation is observed over the course of the first hour after injection.

The results of the observation and measurement of the mucosa elevation were continued up to 14 hours after injection using the device and the compositions as described in Example 1, the results are presented in Table 3, which represents the measurement of the elevation of the mucosae in millimetres (and in %) over the course of the first 14 hours after injection.

Table 3

| Sample | H0 | H6 | H13 | H14 |
|---|---|---|---|---|
| E10 | 4.12 (100%) | 2.07 (50.2%) | 0 | 0 |
| E14 | 6.11 (100%) | 3.82 (62.5%) | 3.22 (52.7%) | 3.22 (52.7%) |
| E15 | 6.79 (100%) | 6.79 (100%) | 5.7 (83.9%) | 5.7 (83.9%) |
| E16 | 4.32 (100%) | 4.32 (100%) | 3.66 (84.7%) | 3.66 (84.7%) |
| E17 | 5.35 (100%) | 5.35 (100%) | 4.76 (88.9%) | 4.76 (88.9%) |
| E18 | 4.94 (100%) | 4.94 (100%) | 3.15 (63.7%) | 3.15 (63.7%) |
| E19 | 5.29 (100%) | 2.78 (52.6%) | 2.06 (39%) | 2.06 (39%) |
| E20 | 4.59 (100%) | 4.41 (96%) | 4.41 (96%) | 4.23 (92.2%) |
| E21 | 2.53 (100%) | 2.53 (100%) | 2.53 (100%) | 2.12 (83.8%) |
| E22 | 4.47 (100%) | 4.47 (100%) | 4.35 (97.3%) | 4.35 (97.3%) |
| E23 | 4.35 (100%) | 4.35 (100%) | 3.23 (74.3%) | 3.23 (74.3%) |
| E24 | 5.23 (100%) | 5.23 (100%) | 5.23 (100%) | 5.23 (100%) |
| E25 | 3.04 (100%) | 2.89 (95.1%) | 2.89 (95.1%) | 2.7 (88.8%) |
| E27 | 4.95 (100%) | 4.23 (85.5%) | 4.23 (85.5%) | 4.23 (85.5%) |
| E28 | 5.56(100%) | 3.79 (68.2%) | 3.79 (68.2%) | 3.79 (68.2%) |

| | | | | |
|---|---|---|---|---|
| E29 | 4.59 (100%) | 3.62 (78.9%) | 3.62 (78.9%) | 3.62 (78.9%) |

Sample 10, which corresponds to SINOVIAL, exhibits a rapid decrease in the mucosa elevation over the course of the first 60 minutes after injection into the submucosal space of the pork bellies compared with samples 14 to 29 of a composition according to the present invention.

One hour after injection, sample 10 exhibits less than 75% of the initial elevation. From the 13th hour after injection, it was noted that the elevation of the mucosa of the samples injected with SINOVIAL (sample 10) had totally disappeared. A cross section of the portion of pork belly was carried out showing a total migration of the product into the submucosal space and the neighbouring tissues, which was visible when the compositions were stained with a few drops of colouring agent making it possible to dye the composition azure blue.

Samples 14 to 29 maintained an elevation greater than that of sample 10. One hour after injection, the samples of a composition according to the present invention maintain an elevation greater than 75% or even, in the majority of cases, equal to 100%. By way of example, samples 14 and 24 exhibit 75.12% and 100% of the initial elevation, 14 hours after injection.

Compositions 14 to 29 from the present invention therefore enable a sustainable elevation of the mucosa while maintaining the state of the resorbable biocompatible polymer compositions in the digestive submucosal space of the portions of pork belly.

Keeping the temperature at a constant 25° C. did not allow the portions of pork belly to be kept for a duration of longer than 1 day, since the portions of pork belly were completely dried out beyond that point.

### Example 2: Progression of the Elevation of the Pork Belly Mucosa after Injection at 4° C.

The conditions and compositions of Example 1 were used while keeping the portions of pork belly at 4° C. under transparent stretch cling film and only removing the samples therefrom in order to take the photographs, while having taken care to mark the cork sheets in order to always reposition them in the same direction and in the same place on the fixed mounting, which can be checked via the sheet of squared paper fixed in the background.

The progression of the collapse of the mucosa elevation over the course of the first 14 hours is identical at 4° C. and 25° C. No mucosal drying out was observed either visually or olfactively.

### Example 3: Progression of the Elevation of the Pork Belly Mucosa Over 4 Days

The experiment was continued over 4 days with the compositions from the present invention (samples E14 to E29) while carrying out photographic prints every twelve hours and keeping the samples at 4° C. under transparent film. Here too, it was possible to observe the perfect freshness of the portions of pork belly both visually and olfactively. The results of the progression of the elevations of the mucosa over the course of the first days are published in FIG. 1 and Table 4. Table 4 below presents the progression as a percentage of the height of the elevation observed with samples E10 to E29, every 12 hours, up to 98 hours after injection.

Table 4

Time after injection (in hours)

| | | | | | | | | | |
|---|---|---|---|---|---|---|---|---|---|
| | 0 | 12 | 24 | 36 | 48 | 60 | 74 | 86 | 98 |
| E10 | 100 | 29.8 | 0 | 0 | 0 | 0 | 0 | 0 | 0 |
| E14 | 100 | 52.7 | 52.7 | 52.7 | 51.2 | 46.2 | 22.4 | 22.4 | 0 |
| E15 | 100 | 84 | 78.8 | 78.8 | 78.8 | 78.6 | 76.8 | 75.4 | 70.8 |
| E16 | 100 | 84.7 | 81.9 | 74.8 | 74.8 | 74.8 | 74.8 | 74.8 | 67.4 |
| E17 | 100 | 89 | 85.8 | 81.9 | 80.2 | 76.1 | 77.9 | 77.8 | 77.8 |
| E18 | 100 | 60 | 57.3 | 57.3 | 57.3 | 52.6 | 57.1 | 53.8 | 53.6 |
| E20 | 100 | 96.1 | 90.4 | 84.5 | 84.5 | 78.2 | 76.9 | 64 | 64 |
| E21 | 100 | 100 | 82.6 | 76.7 | 76.7 | 76.7 | 66.4 | 62.8 | 62.8 |
| E22 | 100 | 97.3 | 86.1 | 85.5 | 85.5 | 80.5 | 60.4 | 60.4 | 60.4 |
| E23 | 100 | 74.3 | 62.1 | 62.1 | 62.1 | 62.1 | 62.1 | 62.1 | 58.2 |
| E24 | 100 | 100 | 86.6 | 83.2 | 80.3 | 76.5 | 76.5 | 66.3 | 60.6 |
| E25 | 100 | 95.1 | 83.2 | 71.7 | 71.7 | 70.7 | 70.7 | 60 | 60 |
| E27 | 100 | 85.5 | 76.6 | 74.7 | 74.7 | 74.7 | 74.7 | 73.7 | 73.7 |
| E28 | 100 | 68.2 | 55.6 | 54.8 | 54.8 | 49.6 | 41.2 | 42.3 | 42.3 |
| E29 | 100 | 78.9 | 65.4 | 59.5 | 59.5 | 56.7 | 56.7 | 56.7 | 56.7 |

Surprisingly, as indicated in FIG. 1 and Table 4, a noticeable maintenance, comprised between 40% and 80% of the initial elevations, was noted up to 4 days after injection of the compositions.

### Example 4: Determination of the Elevation of a Mucosa After Injection of Different Compositions According to the Invention

The following compositions according to the invention were prepared and tested in the portions of pork belly model, these compositions are defined in Table 5 below.

Table 5

| Reference | Composition |
|---|---|
| 1 | 80 parts NaHA 1.2 kDa 6% crosslinked with BDDE, at a concentration of 0.38% (3.8 mg/ml) 15 parts poloxamer P407 with a MW of 12.6 kDa at a concentration of from 15 to 20% 5 parts HPMC at a concentration of 0.2% |
| 2 | 85 parts NaHA 2.4 kDa 3% crosslinked with BDDE, at a concentration of 0.25% 10 parts poloxamer P188 with a MW of 8.4 kDa at a concentration of 10% |
| 3 | 80 parts NaHA 2.4 kDa 5% crosslinked with BDDE, at a concentration of 0.33% 10 parts poloxamer P407 at a concentration of 10% 10 parts polycaprolactone particles with a diameter of from 25 to 50 µm |
| 4 | 25 parts NaHA 2.4 kDa at a concentration of 0.5% 25 parts NaHA 1.2 kDa at a concentration of 0.4% crosslinked with BDDE at R = 15% 50 parts 1% chondroitin sulfate |
| 5 | 70 parts poloxamer P407 with a MW of 12,600 Da at a concentration of 20% |

| | 30 parts polycaprolactone particles with a diameter of from 25 to 50 µm |
|---|---|
| 6 | 70 parts sodium alginate at a concentration of from 0.5 to 1% 30 parts polycaprolactone particles with a diameter of from 25 to 50 µm |
| 7 | 35 parts poloxamer P407 with a MW of 12,600 Da at a concentration of 20 % 35 parts poloxamer P188 at a concentration of 10% 30 parts polycaprolactone particles with a diameter of from 25 to 50 µm |

The compositions above are prepared according to protocols that are well known to a person skilled in the art and are tested in an ex vivo model of a portion of pork belly according to the protocol described in Example 1. These compositions exhibit the same rheological characteristics and the same resorption profiles in the submucosal space as samples E14 to E29.

1. An injectable composition for use thereof in the weight management of a subject, comprising: a sterile isotonic solution of at least one resorbable biocompatible material chosen among polysaccharides and polysaccharide derivatives, said composition having a viscosity comprised between 0.01 Pa.s and 40 Pa.s, said composition being intended to be used in a process which comprises the administration of an effective quantity of said injectable composition into the submucosa of the gastrointestinal tract during an endoscopic procedure in order to obtain a long-lasting elevation of the mucosa for at least 3 hours wherein, in an ex vivo model of pork belly mucosa, after injection of said composition into the submucosa of the intestinal tract, at least 50% of the height of the initial elevation of the mucosa is still present 14 hours after injection.

2. The composition according to claim 1, characterized in that, in an ex vivo model of pork belly mucosa, after injection of said composition into the submucosa of the intestinal tract, at least 50% of the height of the initial elevation of the mucosa is still present 98 hours after injection.

3. The composition according to claim 1, characterized in that said biocompatible material selected from÷ polysaccharides and polysaccharide derivatives, is selected from hyaluronic acid, cellulose, starch, alginic acid, chondroitin and chitosan, polycaprolactone, polyglycolic acid, polylactic acid, homopolymers of lactic acid and purified inverse thermosensitive polymers.

4. The composition according to claim 1, in which said at least one polymer is hyaluronic acid or a salt thereof, with a molecular weight preferably between 1,200 and 2,400 kDa, wherein said hyaluronic acid is selected from: linear hyaluronic acid, crosslinked hyaluronic acid, modified hyaluronic acid and a combination thereof.

5. The composition according to claim 1, comprising a first saline solution of crosslinked hyaluronic acid a having a molecular weight comprised between 900 and 3,500 kDa.

6. The composition according to claim 4, comprising a first saline solution of linear hyaluronic acid having a molecular weight comprised between 900 and 3,500 kDa, and a second saline solution of a hyaluronic acid different from the first solution and having a molecular weight between 900 and 3,500 kDa.

7. The composition according to claim 4, characterized in that said solution comprises between 0.3 and 1% hyaluronic acid.

8. The composition according to claim 1, said composition also comprising at least one agent selected from: an anaesthetic agent, a vasoconstrictor agent, a colouring agent and an antioxidant.

9. A kit comprising at least one composition according to claim 1 and a catheter-type device.

10. A use of a composition according to claim 1 for increasing the feeling of satiety, for limiting the feeling of hunger, for managing excess fat or for managing the weight of a subject.

11. The composition according to claim 5, characterized in that the reticulation degree pf hyaluronic acid is comprised between 1 and 40 %.
